(19) United States
(12) Patent Application Publication
(10) Pub. No.: US 2019/0184210 A1
(43) Pub. Date: Jun. 20, 2019

(54) System and Method for the Pulsed Release of Oxygen for Personal Use

(71) Applicant: Otto J. Placik, Arlington Heights, IL (US)

(72) Inventor: Otto J. Placik, Arlington Heights, IL (US)

(21) Appl. No.: 16/221,443

(22) Filed: Dec. 14, 2018

Related U.S. Application Data
(60) Provisional application No. 62/599,676, filed on Dec. 15, 2017.

(51) Int. Cl. A62B 7/02; A61M 16/10; A61M 16/06; B05B 12/12

(52) U.S. Cl. CPC A62B 7/02; A61M 16/101; B65D 83/262; B05B 12/12; A61M 16/06

(57) ABSTRACT

A system and method for periodically releasing oxygen rich gas for inhalation by a user. A container is filled, at least in part, with oxygen gas. The container has a release valve that can be used to selectively release some of the oxygen gas from the container. An activation unit is provided that is connected to the container. The activation unit operates the release valve at a selected rate. Each periodic pulse contains a volume of the oxygen gas released over a first period of time. The first period of time is preferably no longer than the time it takes a user to take a breath. The periodic pulses are spaced to correspond to the rate of respiration or some multiple thereof. A dispenser is provided that directs the oxygen gas into a place where it can be inhaled.

### RELATED APPLICATIONS

This application claims the benefit of U.S. Provisional Application No. 62/599,676, filed Dec. 15, 2017.

### BACKGROUND OF THE INVENTION

### 1. Field Of The Invention

In general, the present invention relates to systems and methods that automatically control the release of gas from a container when predetermined criteria are met. More particularly, the present invention relates to systems and methods that release measured volumes of oxygen for the benefit of a single person or for improving air quality in a room.

### 2. Prior Art Description

The breathing of oxygen is required for life. For most people, adequate amounts of oxygen can be provided to the body by merely breathing ambient air. However, for some people, the breathing of air is inadequate to provide the oxygen needed by the body. These people require supplemental sources of oxygen, such as oxygen from a canister or an oxygen generator. Depending upon the individual, some people require a constant supply of oxygen, while others require only occasional doses of supplemental oxygen.

Healthy individuals also can benefit from a supplemental oxygen supply. Doses of oxygen can help a person “catch their breath” after exerting their body. This is why many professional athletes dose with supplemental oxygen during breaks in a game. Doses of supplemental oxygen also help in the treatment and prevention of headaches, the treatment of impotence, and the improvement of wound healing. However, breathing supplemental oxygen does have some disadvantages. Oxygen has vasoconstrictive effects on the circulatory system and can reduce peripheral circulation. Oxygen also makes items burn far more rapidly and. intensely. As such, the use of ox gen near any burning object or heat source should be avoided.

It been discovered that many of the benefits of supplemental oxygen can be achieved, and many of the disadvantages avoided, by only using short periodic doses of oxygen. That is, enabling a person to breath regular air most of the time and only occasionally supplementing the air being breathed with a dose of supplemental oxygen. This provides many of the benefits of breathing supplemental oxygen. without causing a fire hazard or causing adverse vasoconstrictive effects.

In the prior art, large volumes of oxygen are packaged in traditional tanks. However, smaller volumes of oxygen are often bottled in pressurized containers, like spray paint, and are sold to the general public. Consumers buy the containers and dispense the oxygen by momentarily depressing a release valve on the container, therein releasing a short burst of oxygen. The trouble with the existing products is that a person must remember to periodically use the container of oxygen in order to obtain the benefits of the oxygen. This is seldom done with any consistency. Rather, as is often. the case, individuals will use the oxygen far too frequently, until the oxygen supply is exhausted, or they will not use the oxygen frequently enough to produce a useful effect.

A need therefore exists for a portable source of oxygen, that is available to consumers, and can provide oxygen in measured periodic doses. This need is met by the present invention as described and claimed below.

### SUMMARY OF THE INVENTION

The present invention is a system and method for periodically releasing oxygen rich gas in a manner that enables the gas to be inhaled by a user. The periodic release rate is coordinated with the user's rate of respiration. As such, the release rate can be as often as one pulse every breath, but is preferably one pulse every few breaths.

The system utilizes a container that is filled, at least in part, with oxygen gas in a concentration greater than that of ambient air. The container has a release valve that can be used to selectively release some of the oxygen gas from the container.

An activation unit is provided that is connected to the container. The activation unit operates the release valve at a selected rate, therein causing periodic pulses of the oxygen gas to be released from the container. Each periodic pulse contains a volume of the oxygen gas released over a first period of time. The first period of time is preferably no longer than the time it takes a user to take a breath. The periodic pulses are spaced to correspond to the rate of respiration or some multiple thereof. For example, one pulse can be provided for every fourth breath.

A dispenser is provided that receives the periodic pulses of oxygen gas being released. The dispenser directs the pulses into an area where the periodic pulses of oxygen gas can be readily inhaled by a user.

### BRIEF DESCRIPTION OF THE DRAWINGS

For a better understanding of the present invention, reference is made to the following description of exemplary embodiments thereof, considered in conjunction with the accompanying drawings, in which:

FIG. 1 is a perspective view of an exemplary embodiment of a dispensing system with one dispenser interface;

FIG. 2 is an exploded view of the exemplary embodiment of a dispensing system shown with a variety of attachable dispensing interfaces;

FIG. 3 is a block diagram schematic showing the components and operation of the activation unit used within the dispensing system; and

FIG. 4 is a perspective view of an alternate exemplary embodiment of a dispensing system for use with a traditional medical supply tank.

### DETAILED DESCRIPTION OF THE DRAWINGS

Although the present invention oxygen pulse system can be adapted for use with many types of commercially sold oxygen canisters and oxygen tanks, only two examples are illustrated and described. The exemplary embodiments are selected in order to set forth two of the best modes contemplated for the invention. The illustrated embodiments, however, are merely exemplary and should not be considered a limitation when interpreting the scope of the appended claims.

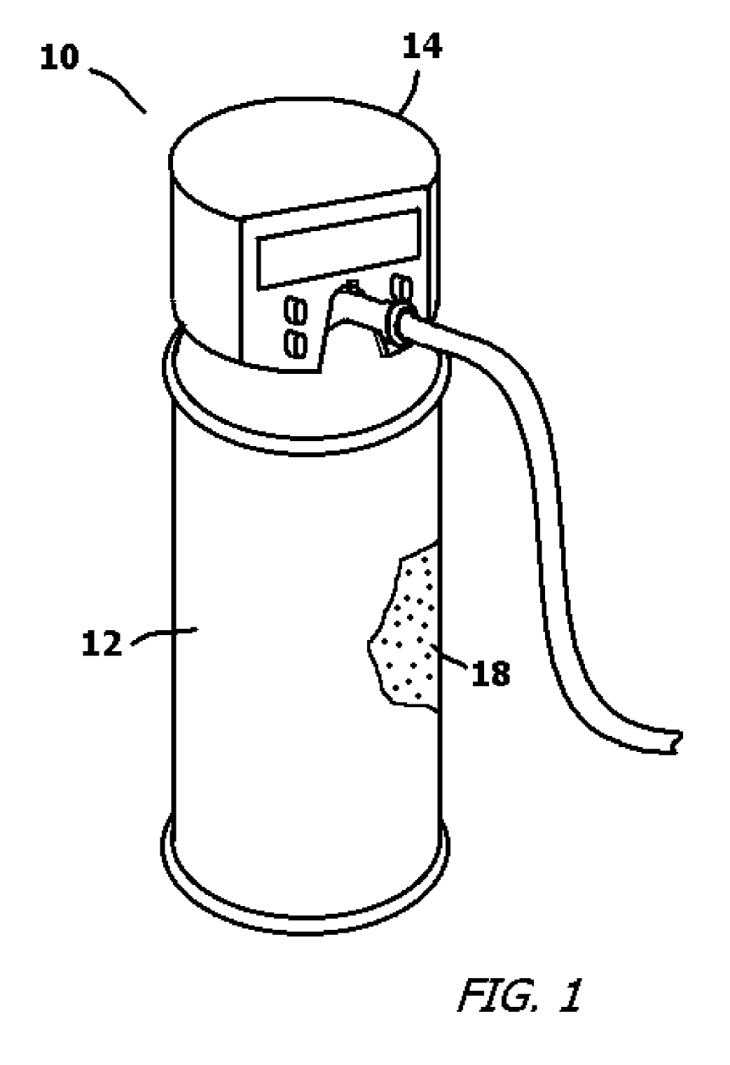

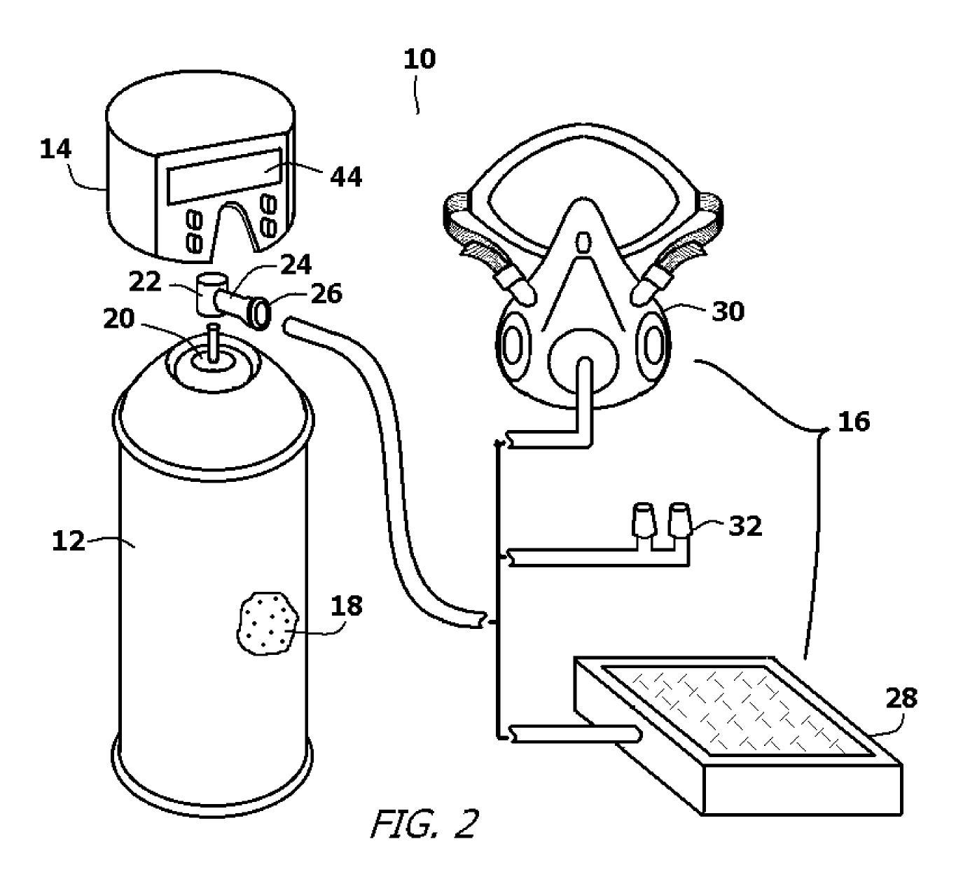

Referring to FIG. 1 in conjunction with FIG. 2, an oxygen pulse system 10 is shown. The oxygen pulse system 10 has three primary components, which include a gas canister 12, an electro-mechanical activation unit 14, and a dispensing interface 16. The purpose of the oxygen pulse system 10 is to dispense a precise dose of oxygen from the gas canister 12 and into the dispensing interface 16 at specific programmed times and/or on demand.

The average person has a respiration rate of between 10 breaths per minute and twenty breaths per minute. The average duration of a breath is between 2 seconds and five seconds, with half that time being dedicated to inhalation and half to exhalation. Additionally, during a breath, an average person inhales approximately 0.5 liters of air. The oxygen pulse system 10 creates pulses of oxygen. Each pulse lasts between 1 second and three seconds, to correspond to the period of time it takes a user to inhale. The pulses preferably occur between every three seconds and thirty seconds. In this manner, the pulses can be coordinated to occur on every breath, every other breath, and up to once every tenth breath. Each pulse releases between 0.05 liters and 0.5 liters of oxygen. In this manner at least 10% of an intake of breath can contain the supplied oxygen.

The gas canister 12 can hold pure oxygen or a combination of compressed gases 18 that includes oxygen and other gases. For example, the gas canister 12 can hold an air/oxygen mix with a higher concentration of oxygen than is present in ambient air. The compressed gases 18 can also contain a small amount of scent so that the dispensing of the compressed gases 18 is more readily perceived by a user. Since the compressed gases 18 are to be inhaled into the body, the compressed gases 18 are sterile and are filtered to meet the appropriate federal and state standards required for inhaled gases.

The compressed gases 18 are held in a traditional gas canister 12 having a release valve 20. Oxygen canisters of this type are commercially available from a variety of manufacturers, such as Boost Oxygen, LLC of Bridgeport, Connecticut. A nozzle 22 is provided that engages the release valve 20. When the nozzle 22 is pressed, the release valve 20 opens and some of the compressed gas 18 is released from the gas cannister 12. The nozzle 22 has a tube connector 24 that extends forward. The tube connector 24 terminates with a tube connection head 26.

The tube connection head 26 can attach to a variety of dispensing interfaces 16. The purpose of the dispensing interface 16 is to channel the released oxygen into an area or position where it can be inhaled by a user. The dispensing interface 16 can be configured as a diffuser 28. The diffuser 28 can be used to diffuse the released compressed gases 18 into a room or some other confined space. The dispensing interface 16 can also be configured as a facemask 30. The facemask 30 can be used to diffuse the compressed gases 18 into the mouth/nose of a person wearing the facemask 30. Likewise, the dispensing interface 16 can also be configured as a breathing tube 32 that can direct the compressed gases 18 into the nose or mouth of a user. Other dispensing interfaces can be used. What is important is that the compressed gases 18 within the gas canister 12 are permitted to diffuse in a controlled manner so that they can be safely inhaled by a user.

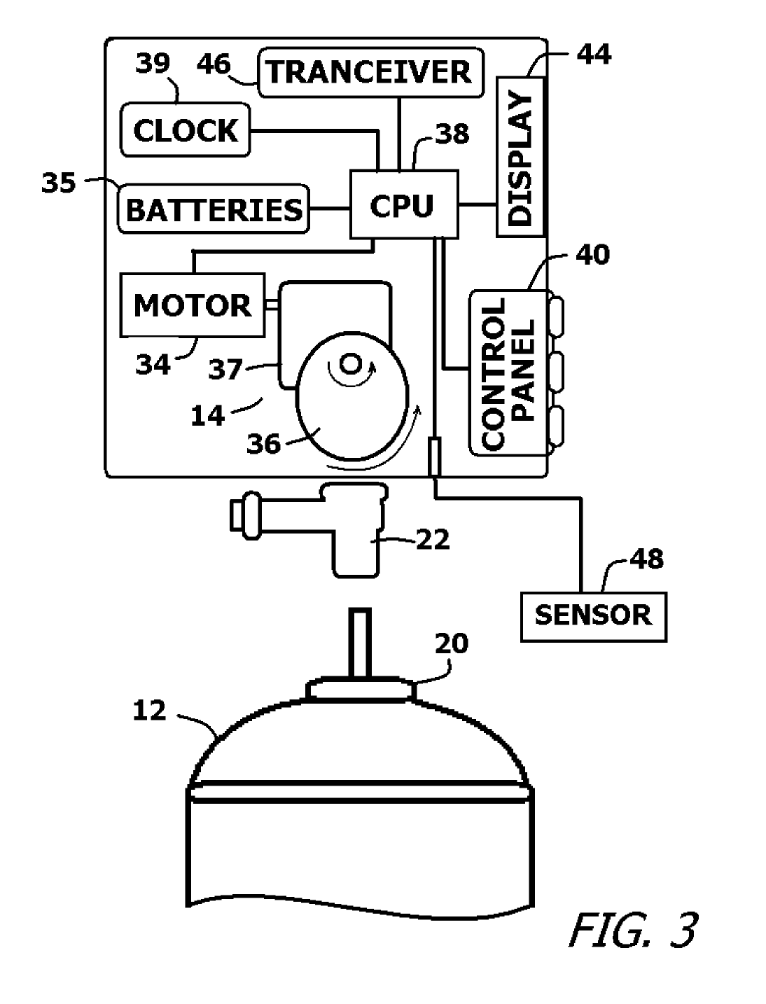

Referring to FIG. 3 in conjunction with FIG. 2, it will be understood that the purpose of the activation unit 14 is to operate the release valve 20 at the top of the gas canister 12. The activation unit 14 is an assembly that attaches to the gas canister 12 over the release valve 20. Within the activation unit 14 there is an electric motor 34 and batteries 35. When activated, the electric motor 34 turns a cam wheel 36. The electric motor 34 can directly turn the cam wheel 36. However, due to size constraints and battery power constraints, it is preferred that a small electric motor be used, wherein the torque provided by the electric motor 34 is increased through the use of a gearbox 37. As the cam wheel 36 turns, the cam wheel 36 contacts and depresses the release valve 20 atop the gas canister 12. The duration of the contact between the cam wheel 36 and the release valve 20 is determined by the shape of the cam wheel 36 and the speed of rotation provided by the electric motor 34 and gearbox 37. It will therefore be understood that the time that the release valve 20 is depressed can be increased or decreased by changing the rotational speed of the electric motor 34, changing the size of the cam wheel 36 and/or changing the input/output ratio of the gearbox 37.

The electric motor 34 is selectively activated and deactivated by a controller 38. The controller 38 can be a dedicated logic circuit or a programable CPU. The controller 38 receives input from an internal clock 39. In this manner, the controller 38 can be programmed to operate the electric motor 34 at various times. The controller 38 is connected to a control panel 40 that enables a person to program the controller 38. The control panel 40 also contains an instant activation button 42 that causes the controller 38 to cycle the electric motor 34 on demand.

The control panel 40 preferably has a display 44 that can display time between cycles and time remaining until the next cycle. The display 44 can also display other useful information, such as the selected rate of discharge, how long the gas canister 12 will last at the selected rate of discharge, the number of discharges made, and/or the number of discharges remaining.

The controller 38 may have the option of being programmed and operated remotely. A wireless transceiver 46, such as a BlueTooth® transceiver, can be included within the activation unit 14. The wireless transceiver 46 enables the controller 38 to communicate with a remove device, such as a smart phone. In this manner, the controller 38 can be programmed through software to run on a user's smartphone.

In use, a user programs the controller 38 with the operational parameters. These may include the duration of a discharge event and the time between discharge events. The duration of the discharge event should be no longer than the time it takes the user to inhale. Otherwise, some of the oxygen released would be wasted. Likewise, the time between discharge events should be coordinated with the respiration rate of the user. At the preprogrammed time of a discharge event, the oxygen pulse system 10 releases a pulse of the compressed gases 18. The duration of the discharge may be set by the manufacturer or may be programed by controlling the rotation rate of the electric motor 34. It is also possible that the oxygen pulse system 10 can be sold with a variety of different interchangeable cam wheels 36. In this manner, the length of a discharge event can be altered by replacing a cam wheel 36. The ability to change the length of the discharge event enables different volumes of the compressed gases 18 to be released during any one release event.

If the selected dispensing interface 16 is a diffuser 28 that vents the compressed gases 18 into an area, then a timed release is all that is required.

However, if the selected dispensing interface 16 is a facemask 30 or a breathing tube 32, then it would be prudent to synchronize a discharge event with the inhalation of a breath by the user. Any discharge event that occurs during the exhalation of a breath may be wasted. To coordinate a discharge event with an inhalation, a sensor 48 is used. The sensor 48 connects to the controller 38 and is used to inform the controller 38 of the cadence of inhalations. The sensor 48 can be either a chest expansion sensor or a pressure sensor. If a chest expansion sensor is used, the sensor 48 is placed on the user's chest and detects when the chest expands and contracts. From the sensor data, the controller 38 can predict the rate of breathing and can delay or advance a scheduled discharge event by a few seconds so that the discharge event occurs at the moment of inhalation. Alternately, the sensor 48 can be a simple pressure sensor that is placed near the nose at the end of a breathing tube 32. The sensor 48 will detect the low pressure during an inhalation and the increased pressure during an exhalation. From the sensor data, the controller 38 can predict the rate of breathing and can delay or advance a scheduled discharge event by a few seconds so that the discharge event occurs at the moment of inhalation.

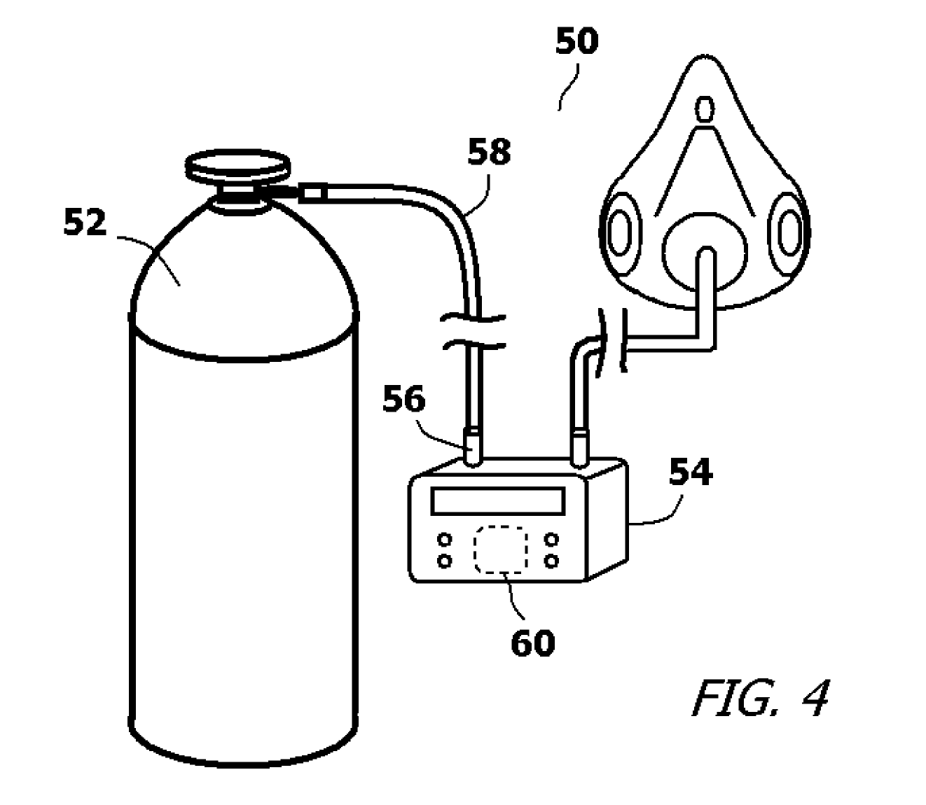

Referring to FIG. 4, an alternate embodiment of the oxygen pulse system 50 is shown for use with a large tank 52 of oxygen gas. The oxygen pulse system 50 has an activation unit 54 with an input port 56. The input port 56 is connected to the oxygen tank 52 with a hose 58 in a traditional manner. The activation unit 54 operates in the same manner as the activation unit 14 in FIG. 3, the only difference being that the prior described motor and cam wheel are replaced by an electronic valve 60. At programmed times, the electronic valve 60 opens and closes to send pulses of oxygen to any dispensing interface 16 that may be connected to the activation unit 54.

It will be understood that the embodiments of the present invention that are illustrated and described are merely exemplary and that a person skilled in the art can make many variations to those embodiments. All such embodiments are intended to be included within the scope of the present invention as defined by the appended claims.

What is claimed is:

1. A system for selectively releasing oxygen rich gas to be inhaled by a user, said system comprising:
a container filled, at least in part, with oxygen in a concentration greater than that of ambient air;
an activation unit connected to said container, wherein said activation unit releases periodic pulses of said oxygen from said container, wherein each of said periodic pulses contains a volume of said oxygen released over a first time period, and wherein each of said periodic pulses is separated by a second time period;
a dispenser that receives said periodic pulses of said oxygen released from said activation unit and directs said periodic pulses into an area where said periodic pulses of said oxygen can be inhaled by a user.

2. The system according to claim 1, wherein said container has a release valve and said activation unit has an electro-mechanical assembly that temporarily opens said release valve when activated.

3. The system according to claim 1, wherein said dispenser is selected from a group consisting of facemasks, breathing tubes and room diffusers.

4. The system according to claim 1, wherein said volume of said volume released is between 0.1 liters and 0.5 liters.

5. The system according to claim 1, wherein said periodic pulses occur between once every 10 seconds and once every twenty seconds.

6. The system according to claim 1, wherein said first period of time is between one second and four seconds.

7. The system according to claim 1, wherein said activation unit contains an electronic controller and a transceiver that enables said electronic controller to receive commands from a remote computing device.

8. The system according to claim 1, wherein said activation unit contains a programable controller and a control panel that enables a user to program said programable controller.

9. The system according to claim 1, further including a sensor for detecting a rate or respiration of a user, wherein said sensor is coupled to said activation unit, wherein said activation unit automatically alters said second period of time to correspond to said respiration rate detected by said sensor.

10. A system for selectively releasing oxygen rich gas to be inhaled by a user, said system comprising:
an oxygen canister filled primarily with oxygen gas;
a nozzle that releases some of said oxygen gas from said oxygen canister when depressed;
a mechanism that temporarily depresses said nozzle when activated, therein releasing a volume of said oxygen gas from said oxygen container;
an electronic controller that activates said mechanism at a selected rate, therein causing some of said oxygen gas to be periodically released at said selected rate;
a dispenser that receives said oxygen gas released through said nozzle, wherein said dispenser directs said oxygen gas into an area where said oxygen gas can be inhaled by a user.

11. The system according to claim 10, wherein said mechanism includes a motor that tuns a cam, wherein said cam temporarily depresses said nozzle as said cam is rotated by said motor.

12. The system according to claim 10, wherein said dispenser is selected from a group consisting of facemasks, breathing tubes and room diffusers.

13. The system according to claim 10, wherein said volume of said oxygen gas released is between 0.1 liters and 0.5 liters.

14. The system according to claim 10, wherein said selected rate is between once every ten seconds and once every twenty seconds.

15. The system according to claim 10, wherein said mechanism temporarily depresses said nozzle for a period of time between one second and four seconds when activated.

16. The system according to claim 10, further including a transceiver that enables said electronic controller to receive commands from a remote computing device.

17. The system according to claim 10, further including a control panel that enables a user to program said electronic controller.

18. The system according to claim 10, further including a sensor for detecting a rate or respiration of a user, wherein said sensor is coupled to said electronic controller, wherein said electronic controller operates said mechanism at a rate that corresponds to said respiration rate detected by said sensor.
